(19) United States
(12) Patent Application Publication
(10) Pub. No.: US 2003/0153773 A1
(43) Pub. Date: Aug. 14, 2003

(54) Process for the production of dihydropyridines

(76) Inventors: Gregory S. Wayne, Vernon Hills, IL (US); Wenke Li, Gurnee, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/075,363

(22) Filed: Feb. 14, 2002

(51) Int. Cl. C 07D 4 5/02; C07D 213/14

(52) U.S. Cl. 549/415; 546/250

(57) ABSTRACT

The present invention relates to the production of a salt of a bis-condensation reaction.

### BACKGROUND OF THE INVENTION

Potassium channels play an important role in regulating cell membrane excitability. When the potassium channels open, changes in the electrical potential across the cell membrane occur and result in a more polarized state. A number of diseases or conditions may be treated with therapeutic agents that open potassium channels; see for example (K. Lawson, Pharmacol. Ther., v. 70, pp.39-63 (1996)); (D. R. Gehiert et al., Prog. Neuro-Psychopharmacol & Biol. Psychiat., v. 18, pp. 1093-1102 (1994)); (M. Gopalakrishnan et al., Drug Development Research, v. 28, pp. 95-127 (1993)); (J. E. Freedman et al., The Neuroscientist, v. 2, pp. 145-152 (1996)); (D. E. Nurse et al., Br. J. Urol., v. 68 pp. 27-31 (1991)); (B. B. Howe et al., J. Pharmacol. Exp. Ther., v. 274 pp. 884-890 (1995)); (D. Spanswick et al., Nature, v. 390 pp. 521-25 (Dec. 4, 1997)); (Dompeling Vasa. Supplementum (1992) 3434); (WO9932495); (Grover, J Mol Cell Cardiol. (2000) 32, 677); and (Buchheit, Pulmonary Pharmacology & Therapeutics (1999) 12, 103). Such diseases or conditions include asthma, epilepsy, male sexual dysfunction, female sexual dysfunction, pain, bladder overactivity, stroke, diseases associated with decreased skeletal blood flow such as Raynaud's phenomenon and intermittent claudication, eating disorders, functional bowel disorders, neurodegeneration, benign prostatic hyperplasia (BPH), dysmenorrhea, premature labor, alopecia, cardioprotection, coronary artery disease, angina, ischemia, and incontinence.

Production of dihydropyridine potassium channel openers typically calls for the reaction of a diketone and an aldehyde with an ammonia source, such as ammonium hydroxide. This procedure involves a difficult purification due to the product's low solubility. The present invention involves the isolation of a salt of a bis-condensation product that is important in that it has different solubility properties as compared to the dihydropyridine product, allowing for purification. The bis-condensation product occurs via a rare mechanistic pathway (Katritzky, A. et al., Tetrahedron, 1986, 42, 5729-5738).

### DETAILED DESCRIPTION OF THE INVENTION

The present invention involves a novel process and novel intermediates for producing dihydropyridine compounds that are useful as potassium channel openers. In particular, the present invention relates to isolation of a salt of a bis-condensation product. The salt allows for easy isolation and purification as compared to the dihydropyridine product.

In one embodiment of the present invention as shown in Scheme 1, the process involves reacting two equivalents of a diketone (1) and one equivalent of an aldehyde (2) in the presence of a base in a solvent. Suitable bases for use in the present invention include, but are not intended to be limited to, tertiary amine bases, pyridine, DBU (1,9-diazabicyclo[5.4.0]undec-7-ene) and DBN (1,5-diazabicyclo[4.3.0]non-5-ene). A more preferred base is triethylamine or diisopropylethylamine. Suitable solvents for use in the present invention include alcohol solvents. A more preferred solvent is a 1:1 mixture of ethyl acetate and isopropanol.

The bis-condensation product precipitates out as the triethylamine salt (3) which may then be reacted with ammonium actetate in acetic acid at high temperature to yield the dihydopyridine (5), which precipitates out of solution. In Scheme 1, R is selected from the group consisting of substituted and unsubstituted aryl and heterocycle.

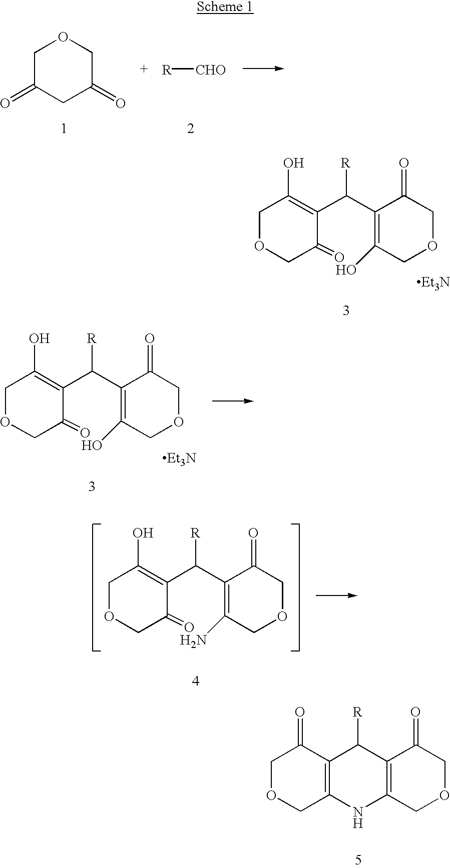

A preferred embodiment of the present invention as shown in Scheme 1, wherein R is 3-iodo-4-fluorophenyl, 3,5-dioxopyran (1) and 3-iodo-4-fluoro-benzaldehyde are reacted together in base and solvent to form the 4-fluoro-3-iodo-bis-(3,5-dioxo-tetrahydro-pyran-4-yl)-methane triethylamine salt (3). The triethylamine salt is then reacted with ammonium acetate in the presence of acetic acid and heat to produce the dihydropyridine 5-(4-fluro-3-iodophenyl)-5,10dihydro-1H3H-dipyrano[3,4-b:4,3-e]pyridine-4,6(7H1,9H)dione.

The reaction of ammonium acetate is a preferred method of producing the dihydropyridine product in that it is a fast reaction and relatively free of impurities. The major impurity obtained is the pyran derivative. The pyran impurity may be removed by dissolving the product in an aqueous potassium hydroxide/ethanol solution to hydrolyze the pyran impurity to the open form which remains in the liquids upon pH adjustment and dihydropyri dine precipitation.

The term “aryl” as used herein, means a phenyl group, or a bicyclic or a tricyclic fused ring system wherein one or more of the fused rings is a phenyl group. Bicyclic fused ring systems are exemplified by a phenyl group fused to a cycloalkyl group, as defined herein, or another phenyl group. Tricyclic fused ring systems are exemplified by a bicyclic fused ring system fused to a cycloalkyl group, as defined herein, or another phenyl group. Representative examples of aryl include, but are not limited to, anthracenyl, azulenyl, fluorenyl, indanyl, indenyl, naphthyl, phenyl and tetrahydronaphthyl.

The aryl groups of this invention can be substituted with 1, 2, 3, 4 or 5 substituents independently selected from alkenyl, alkoxy, alkyl, alkynyl, carboxy, cyano, formyl, haloalkyl, halogen, hydroxy, hydroxyalkyl, and nitro.

The term “heterocycle” or “heterocyclic” as used herein, means a monocyclic, bicyclic, or tricyclic ring system. Monocyclic ring systems are exemplified by any 3-or 4-membered ring containing a heteroatom independently selected from oxygen, nitrogen and sulfur; or a 5-, 6-or 7-membered ring containing one, two or three heteroatoms wherein the heteroatoms are independently selected from nitrogen, oxygen and sulfur. The 5-membered ring has from 0-2 double bonds and the 6-and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzofuranyl, isobenzothienyl, isoindolyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl. The heterocycles of this invention can be substituted with 1, 2,or 3 substituents independently selected from from alkenyl, alkoxy, alkyl, alkynyl, carboxy, cyano, formyl, haloalkyl, halogen, hydroxy, hydroxyalkyl, and nitro.

### EXAMPLE 1

To a 50 mL flask, pyran-3,5-dion (2 g) and 4-fluoro-3-iodobenzaldehyde (2.19 g) were added. Ethyl acetate (8 mL) and isopropanol (8 mL) were added, followed by triethylamine (1.22 mL). The reaction mixture was heated to 50° C. and stirred for 1 hour. The resultant slurry was cooled to 2° C. and then filtered. The wetcake was washed with cold isopropanol/ethyl acetate (10 mL; 1:1) and then dried in the vacuum oven at 65° C. Obtained 4.00 g product. Spectral Data: ¹H NMR (300 MHz/CDC1₃) δ7.48-7.51 (m,1H), 7.12-7.18 (m, 1H), 6.88(t, J=8 Hz, 1H), 5.90 (s, 1H), 4.17 (br s, 8H), 3.18 (q, J=7 Hz, 6H), 1.23 (t,J=7 Hz, 9H).

### EXAMPLE 2

To a 50 mL flask was charged 4-fluoro-3-iodo-bis-(3,5-dioxo-tetrahydro-pyran-4-yl)-methane triethylamine salt (5.0 g), acetic acid (25 mL) and distilled water (0.5 mL). Then ammonium acetate (3.43 g) was added and the reaction mixture was heated to 105° C. and stirred at this temperature for 1 hour. The reaction mixture was then cooled to 25° C. and filtered. The wetcake was washed with ethanol (25 mL) and air-dried on the filter to give 3.45 g crude product.

### EXAMPLE 3

A solution was made up consisting of ethanol (210 mL), water (23 mL) and potassium hydroxide (2.34 g). This was added to the dihydropyridine (12.0 g) and stirred to dissolve everything. After cooling to 10-15° C., 0.4 M hydrochloric acid was added slowly. Once the pH reached below 7, the resulting slurry was filtered and the wetcake washed with ethanol/water (63 mL; 2.5:1), followed by ethanol (32 mL). The wetcake was dried in the vacuum oven to give 12.37 g product.

We claim

1. A process for producing a salt of a bis-condensation reaction product comprising reacting a diketone and an aldehyde in the presence of a base and a solvent.

2. A process of claim 1 wherein said base is selected from the group consisting of tertiary amine base, pyridine, DBU and DBN.

3. A process of claim 2 wherein said tertiary amine base is triethylamine.

4. A process of claim 1 wherein said solvent is isopropanol, ethyl acetate or mixtures of isopropanol and ethyl acetate.

5. A compound and salts thereof of formula 3, wherein R is selected from the group consisting of substituted and unsubstituted aryl and heterocycle.

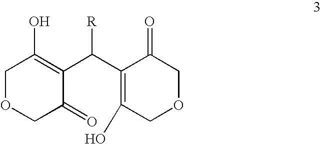

6. A compound of claim 5 wherein R is 3-iodo-4-fluorophenyl.

7. A process for removing dihydropyran from dihydropyridine comprising dissolving dihydropyridine in a mixture of aqueous base and an organic solvent, followed by acidification.

8. A process of claim 7 wherein said organic solvent is ethanol.

9. A process of claim 7 wherein said aqueous base is selected from the group consisting of potassium hydroxide and sodium hydroxide.
